(19) United States
(12) Patent Application Publication
(10) Pub. No.: US 2025/0127831 A1
(43) Pub. Date: Apr. 24, 2025

(54) STRAIN OF LACTICASEIBACILLUS PARACASEI CAPABLE OF RELIEVING HYPERSENSITIVE RHINITIS AND APPLICATION THEREOF

(71) Applicant: Shenzhen Porshealth Bioengineering Co., Ltd., Shenzhen (CN)

(72) Inventors: Silu Zhang, Shenzhen (CN); Yanyi Zheng, Shenzhen (CN); Guoxun Xiao, Shenzhen (CN); Xiaoli Zhou, Shenzhen (CN); Zhizhu Zhang, Shenzhen (CN)

(21) Appl. No.: 18/954,554

(22) Filed: Nov. 21, 2024

(30) Foreign Application Priority Data
Nov. 21, 2023 (CN) 202311561656.6

(51) Int. Cl. A61K 35/747; A61K 47/38; A61K 47/54; A61P 11/02; A61P 37/08

(52) U.S. Cl. CPC A61K 35/747; A61K 47/38; A61K 47/544; A61P 11/02; A61P 37/08

(57) ABSTRACT

A strain of Lacticaseibacillus paracasei capable of relieving hypersensitive rhinitis and its application are disclosed, belonging to the technical field of biological medicines. Lacticaseibacillus paracasei GOLDGUT-Lpc969 can obviously relieve the symptoms of hypersensitive rhinitis of mice, reduce the IgE content in serum of the mice with hypersensitive rhinitis, reduce the histamine content in serum of the mice with hypersensitive rhinitis, reduce the content of allergic cytokines in serum of the mice with hypersensitive rhinitis, increase Th1-type immune response activity in mice with hypersensitive rhinitis, reduce the levels of pro-inflammatory cytokines in serum of mice with hypersensitive rhinitis, repair lesions of nasal mucosa of mice with hypersensitive rhinitis, reduce histamine concentration released by RBL-2H3 cells induced by anti-DNP-IgE, reduce the degree of degranulation of RBL-2H3 cells induced by anti-DNP-IgE, and inhibit release of inflammatory cytokines induced by LPS, which has application prospect in preparing products for preventing and/or treating hypersensitive rhinitis.

### SEQUENCE LISTING

The sequence listing is submitted as a XML file filed via EFS-Web, with a file name of “Substitute_Sequence_Listing.XML”, a creation date of Dec. 21, 2024, and a size of 5741 bytes. The sequence Listing filed via EFS-Web is a part of the specification and is incorporated in its entirety by reference herein.

### TECHNICAL FIELD

The disclosure relates to a strain of Lacticaseibacillus paracasei capable of relieving hypersensitive rhinitis and its application, which belongs to the technical field of biological medicines.

### BACKGROUND ART

Hypersensitive rhinitis is a non-infectious inflammatory disease of the nasal mucosa, which is one of allergies, and its symptoms are similar to a cold. The main symptoms of hypersensitive rhinitis are sneeze, followed by eye itching, intranasal itching, nasal obstruction, runny nose, clear water like white nasal discharge and the like, which occur intermittently and repeatedly. At the onset, the nasal mucosa is pale and edematous. If there is an allergic constitution, it will cause urticaria. Severe conditions may also evolve into sinusitis, asthma, or ear infections.

There is no complete cure for hypersensitive rhinitis, and symptoms are generally relieved through drugs or other methods. For example, cetirizine hydrochloride and loratadine are prescribed as antihistamines for patients at the time of onset. Hormonal agents such as corticosteroids may also reduce the number of episodes of hypersensitive rhinitis or reduce its allergic response. The anti-hyperemic agent can relieve hyperemia and edema of nasal mucosa, which improves nasal obstruction symptoms of hypersensitive rhinitis.

However, the traditional antihistamine medicament can cause side effects of sleepiness, mental fatigue, headache, constipation and the like of patients, the hormone medicament can also easily cause side effects of endocrine dyscrasia, immunity decline, osteoporosis and the like, and the anti-hyperemic agent can cause the occurrence of blood pressure rise, angina pectoris attack, arrhythmia, migraine, ischemic stroke and the like. Therefore, it is urgent to find a therapeutic agent for hypersensitive rhinitis which has lower side effects, is more effective and is less prone to immune tolerance.

### SUMMARY

In order to solve the above problems, the disclosure provides a strain of Lacticaseibacillus paracasei GOLDGUT-Lpc969, wherein the strain of Lacticaseibacillus paracasei GOLDGUT-Lpc969 was deposited in China General Microbiological Culture Collection Center (CGMCC) with a deposit number of CGMCC No. 28149 and a deposit date of Aug. 10, 2023.

The Lacticaseibacillus paracasei GOLDGUT-Lpc969 was derived from fresh fecal samples of healthy people in Shenzhen. After sequencing and analysis, the 16S rDNA sequence of the strain was shown in SEQ ID NO. 1. The sequence obtained by sequencing was subjected to nucleic acid sequence alignment in Blastn program at NCBI, and the result showed that the strain was Lacticaseibacillus paracasei and was named as Lacticaseibacillus paracasei GOLDGUT-Lpc969.

The disclosure also provides application of the above Lacticaseibacillus paracasei in preparing a drug for a prevention and/or a treatment of hypersensitive rhinitis.

In one embodiment of the present disclosure, the prevention and/or the treatment of hypersensitive rhinitis include alleviation of symptoms of hypersensitive rhinitis, inhibition of immune responses caused by hypersensitive rhinitis, inhibition of inflammatory responses caused by hypersensitive rhinitis and/or repair of lesions of nasal mucosa caused by hypersensitive rhinitis.

In one embodiment of the present disclosure, the alleviation of symptoms of hypersensitive rhinitis includes reducing the frequency of nasal scratching, sneezing and/or nasal discharge in patients with hypersensitive rhinitis.

In one embodiment of the present disclosure, in the drug, the viable count of the above-mentioned Lacticaseibacillus paracasei GOLDGUT-Lpc969 is not less than 1×10⁶ CFU/mL or 1×10⁶ CFU/g.

In one embodiment of the present disclosure, the drug includes the above-described Lacticaseibacillus paracasei GOLDGUT-Lpc969, a drug carrier and/or a pharmaceutical adjuvant.

In one embodiment of the present disclosure, the drug carrier includes a microcapsule, a microsphere, a nanoparticle and/or a liposome.

In one embodiment of the present disclosure, the pharmaceutical adjuvant includes an excipient and/or an additive.

In one embodiment of the present disclosure, the excipient includes a solvent, a propellant, a solubilizer, a cosolvent, an emulsifier, a colorant, an absorbent, a diluent, a flocculant, a deflocculant, a filter aid, and/or a release retardant.

In one embodiment of the present disclosure, the additive includes microcrystalline cellulose, hydroxypropyl methylcellulose, and/or refined lecithin.

In one embodiment of the present disclosure, the dosage form of the drug includes a powder, a granule, a capsule, a tablet, a pill or an oral liquid.

The disclosure also provides a product containing the above-mentioned Lacticaseibacillus paracasei GOLDGUT-Lpc969.

In one embodiment of the present disclosure, in the product, the viable count of the above-mentioned Lacticaseibacillus paracasei GOLDGUT-Lpc969 is not less than 1×10⁶ CFU/mL or 1×10⁶ CFU/g.

In one embodiment of the present disclosure, the product includes a food or drug; the food is special medical food, health care product or functional beverage.

In one embodiment of the present disclosure, the drug includes the above-described Lacticaseibacillus paracasei GOLDGUT-Lpc969, a drug carrier and/or a pharmaceutical adjuvant.

In one embodiment of the present disclosure, the drug carrier includes a microcapsule, a microsphere, a nanoparticle and/or a liposome.

In one embodiment of the present disclosure, the pharmaceutical adjuvant includes an excipient and/or an additive.

In one embodiment of the present disclosure, the excipient includes a solvent, a propellant, a solubilizer, a cosolvent, an emulsifier, a colorant, an absorbent, a diluent, a flocculant, a deflocculant, a filter aid, and/or a release retardant.

In one embodiment of the disclosure, the additive includes microcrystalline cellulose, hydroxypropyl methylcellulose, and/or refined lecithin.

In one embodiment of the disclosure, the dosage form of the drug includes a powder, a granule, a capsule, a tablet, a pill or an oral liquid.

In one embodiment of the disclosure, the food contains the above-described Lacticaseibacillus paracasei GOLDGUT-Lpc969 and/or a food additive.

In one embodiment of the disclosure, the food additive includes an antioxidant, a bleach, a colorant, a color fixative, an enzyme preparation, a flavoring enhancer, a preservative, and/or a sweetener.

The technical scheme of the disclosure has the following advantages: the disclosure provides a strain of Lacticaseibacillus paracasei GOLDGUT-Lpc969, with a deposit number of CGMCC No.28149. The strain of Lacticaseibacillus paracasei GOLDGUT-Lpc969 can obviously relieve the symptoms of hypersensitive rhinitis of mice (namely, reduce the frequency of nasal scratching, sneezing and/or nasal discharge in mice with hypersensitive rhinitis, can obviously reduce the IgE content in serum of the mice with hypersensitive rhinitis, can obviously reduce the histamine content in serum of the mice with hypersensitive rhinitis, can obviously reduce the content of allergic cytokines (IL-2, IL-4, IL-5 and IL-13) in serum of the mice with hypersensitive rhinitis, can significantly increase Th1-type immune response activity in mice with hypersensitive rhinitis (i.e. increase IFN-gamma/IL-4 ratio in serum of mice with hypersensitive rhinitis), can significantly reduce the levels of pro-inflammatory cytokines (IL-6 and THF-α) in serum of mice with hypersensitive rhinitis, can significantly repair lesions of nasal mucosa of mice with hypersensitive rhinitis, can significantly reduce histamine concentration released by RBL-2H3 cells induced by anti-DNP-IgE, can significantly reduce the degree of degranulation of RBL-2H3 cells induced by anti-DNP-IgE, and can significantly inhibit release of inflammatory cytokines (IFN-γ, IL-10, IL-2, IL-4, IL-6 and THF-α) induced by LPS. It can be seen that, the Lacticaseibacillus paracasei GOLDGUT-Lpc969 has great application prospect in preparing products (such as food, drug and the like) for preventing and/or treating hypersensitive rhinitis.

In addition, the Lacticaseibacillus paracasei is one of probiotics, which has been incorporated into the edible probiotic list of China at present, so that the Lacticaseibacillus paracasei GOLDGUT-Lpc969 and the product with the Lacticaseibacillus paracasei GOLDGUT-Lpc969 as the active ingredients of the present disclosure have the advantages of small side effect, low administration threshold of patients, difficult immune tolerance generation and lower manufacturing cost, with large development potential and good market prospect.

### Deposit of Biological Material

A strain of Lacticaseibacillus paracasei GOLDGUT-Lpc969, having a taxonomic designation of Lacticaseibacillus paracasei, was deposited on Aug. 10, 2023 at the China General Microbiological Culture Collection Center, with a deposit number of CGMCC No. 28149 and a deposit address of No. 3, Courtyard 1, West Beichen Road, Chaoyang District, Beijing.

### BRIEF DESCRIPTION OF THE DRAWINGS

FIG. 1: effect of Lacticaseibacillus paracasei GOLDGUT-Lpc969 on DAI score of mice with OVA-induced hypersensitive rhinitis.

FIG. 2: effect of Lacticaseibacillus paracasei GOLDGUT-Lpc969 on serum IgE levels of mice with OVA-induced hypersensitive rhinitis.

FIG. 3: effect of Lacticaseibacillus paracasei GOLDGUT-Lpc969 on serum histamine levels of mice with OVA-induced hypersensitive rhinitis.

FIG. 4: effect of Lacticaseibacillus paracasei GOLDGUT-Lpc969 on serum IL-2 concentration of mice with OVA-induced hypersensitive rhinitis.

FIG. 5: effect of Lacticaseibacillus paracasei GOLDGUT-Lpc969 on serum IL-4 concentration of mice with OVA-induced hypersensitive rhinitis.

FIG. 6: effect of Lacticaseibacillus paracasei GOLDGUT-Lpc969 on serum IL-5 concentration of mice with OVA-induced hypersensitive rhinitis.

FIG. 7: effect of Lacticaseibacillus paracasei GOLDGUT-Lpc969 on serum IL-13 concentration of mice with OVA-induced hypersensitive rhinitis.

FIG. 8: effect of Lacticaseibacillus paracasei GOLDGUT-Lpc969 on serum IFN-gamma/IL-4 ratio of mice with OVA-induced hypersensitive rhinitis.

FIG. 9: effect of Lacticaseibacillus paracasei GOLDGUT-Lpc969 on serum IL-6 concentration of mice with OVA-induced hypersensitive rhinitis.

FIG. 10: effect of Lacticaseibacillus paracasei GOLDGUT-Lpc969 on serum TNF-α concentration of mice with OVA-induced hypersensitive rhinitis.

FIG. 11: effect of Lacticaseibacillus paracasei GOLDGUT-Lpc969 on nasal mucosa pathology of mice with OVA-induced hypersensitive rhinitis.

FIG. 12: effect of Lacticaseibacillus paracasei GOLDGUT-Lpc969 on histamine secretion in RBL-2H3 cells induced by anti-DNP-IgE.

FIG. 13: effect of Lacticaseibacillus paracasei GOLDGUT-Lpc969 on degranulation release in RBL-2H3 cells induced by anti-DNP-IgE.

FIG. 14: effect of Lacticaseibacillus paracasei GOLDGUT-Lpc969 on LPS-induced secretion of IFN-γ by human PBMC.

FIG. 15: effect of Lacticaseibacillus paracasei GOLDGUT-Lpc969 on LPS-induced secretion of IL-10 by human PBMC.

FIG. 16: effect of Lacticaseibacillus paracasei GOLDGUT-Lpc969 on LPS-induced secretion of IL-2 by human PBMC.

FIG. 17: effect of Lacticaseibacillus paracasei GOLDGUT-Lpc969 on LPS-induced secretion of IL-4 by human PBMC.

FIG. 18: effect of Lacticaseibacillus paracasei GOLDGUT-Lpc969 on LPS-induced secretion of IL-6 by human PBMC.

FIG. 19: effect of Lacticaseibacillus paracasei GOLDGUT-Lpc969 on LPS-induced secretion of TNF-α by human PBMC.

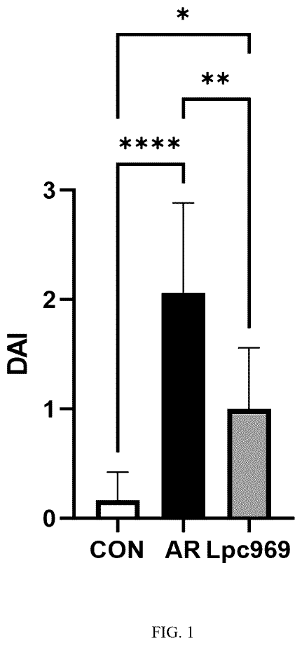

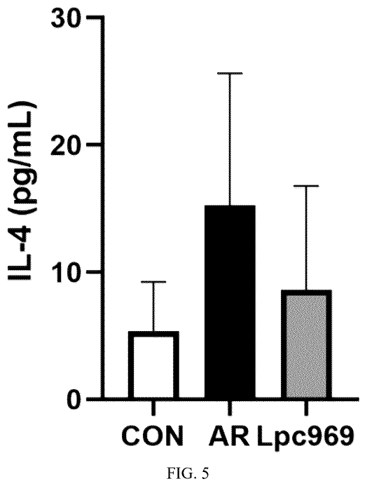

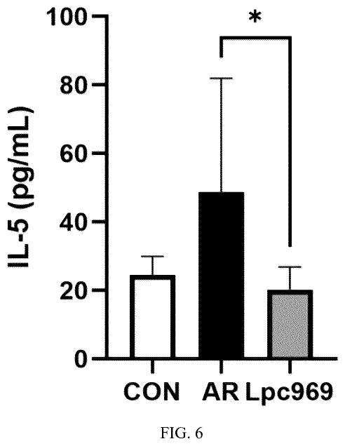

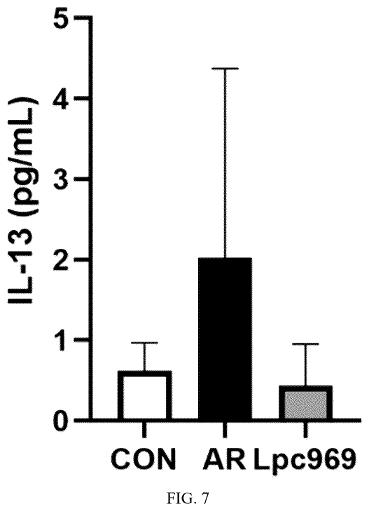

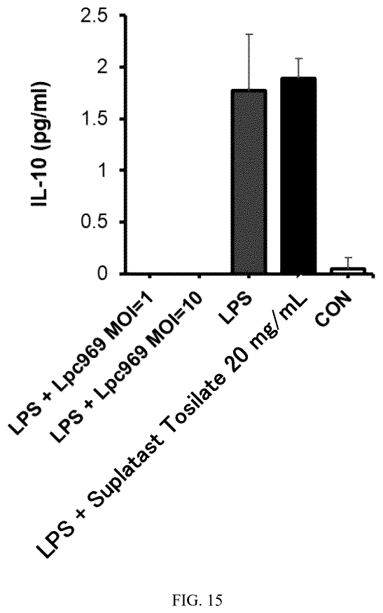

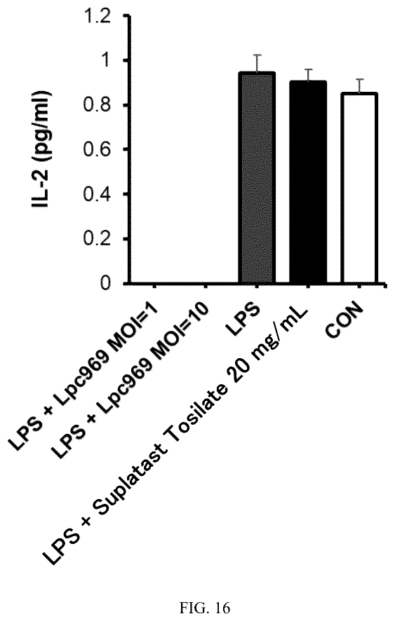

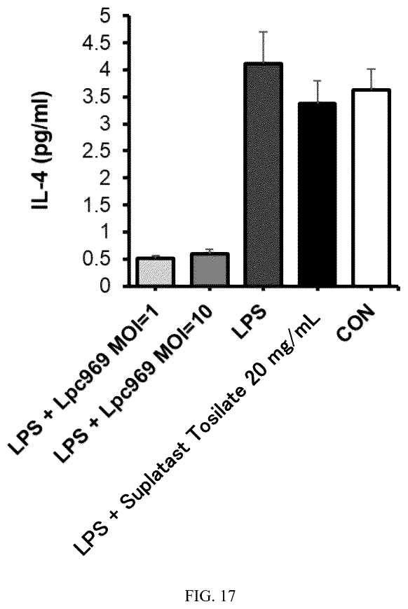

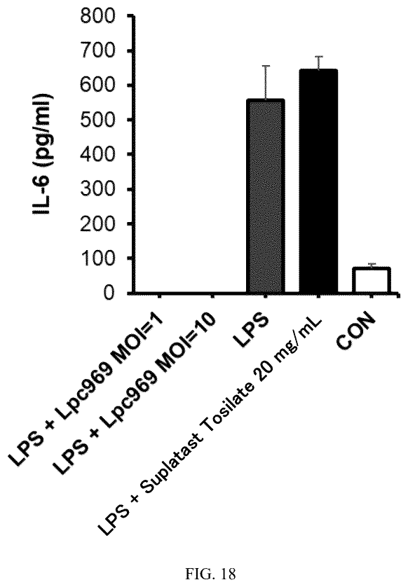

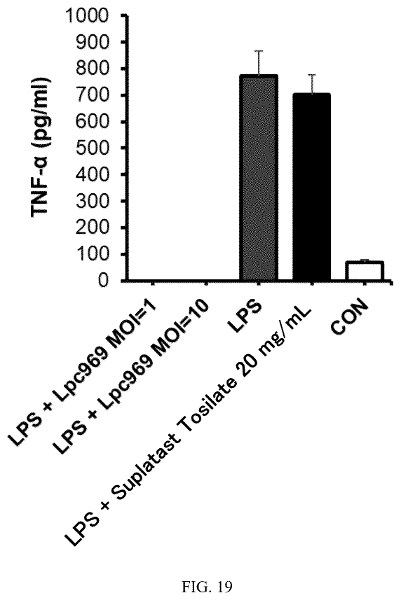

In FIG. 1-FIG. 19, *p<0.05, ** p<0.01, *** p<0.001, **** p<0.0001.

### DETAILED DESCRIPTION OF THE EMBODIMENTS

The following embodiments are provided to better understand the present disclosure, are not limited to the best embodiments, and do not limit the content and scope of protection of the present disclosure. Any product identical or similar to the present disclosure obtained by anyone under the inspiration of the present disclosure or by combining the present disclosure with other prior art features will fall within the scope of protection of the present disclosure.

### The Following Embodiments Relate to the Following Media

MRS solid medium: peptone 10 g/L, beef extract powder 5 g/L, yeast extract powder 4 g/L, glucose 20 g/L, sodium acetate 5 g/L, dipotassium hydrogen phosphate 2 g/L, triammonium citrate 2 g/L, magnesium sulfate 0.2 g/L, manganese sulfate 0.05 g/L, Tween 80 1 g/L, agar 15 g/L, cysteine ammonia salt 0.5 g/L, pH 6.8.

MRS liquid medium: peptone 10 g/L, beef extract powder 5 g/L, yeast extract powder 4 g/L, glucose 20 g/L, sodium acetate 5 g/L, dipotassium hydrogen phosphate 2 g/L, triammonium citrate 2 g/L, magnesium sulfate 0.2 g/L, manganese sulfate 0.05 g/L, Tween 80 1 g/L, cysteine ammonia salt 0.5 g/L, pH 6.8.

### Embodiment 1

Acquisition of Lacticaseibacillus paracasei

The method included the following specific steps:

Fresh feces of healthy people from Shenzhen region were taken as a sample, 0.5 mL of the sample was taken, added into 5 mL of MRS liquid medium, and cultured for 24 h at 37° C. for enrichment to obtain an enriched sample; the 0.5 mL of the enriched sample was pipetted into 4.5 mL of sterile physiological saline to obtain a 10⁻diluent and then 0.5 mL of the 10⁻¹ diluent was pipetted into 4.5 mL of physiological saline to obtain a 10⁻² diluent, then this procedure was followed to obtain 10⁻³, 10⁻⁴, 10⁻⁵ and 10⁻⁶ diluents in turn; 100 μL of the gradient diluent was pipetted and coated on the MRS solid medium with one plate for each gradient of 10⁻⁴, 10⁻⁵ and 10^{-6,} the same was incubated at 37° C. for 48 h to obtain colonies; the colonies with typical characteristics of Lacticaseibacillus paracasei on the MRS solid medium were selected according to the shape, size, edge, transparency and the like of the colonies, then the selected colonies were picked out with the inoculation ring and streaked on the MRS solid medium, and cultured at 37° C. for 48 h to obtain purified single colonies; the purified single colonies were selected and inoculated into 5 mL MRS liquid medium respectively, and cultured at 37° C. for 24 h to obtain bacterial liquids; after numbering each strain corresponding to each bacterial liquid, Gram staining, strain identification, physiological and biochemical experiments and genome identification analysis were performed by referring to the steps described in textbook “Microbiology” (edited by Shen Ping and Chen Xiangdong), and hemolytic activity test was performed by referring to the steps described in 3.7 of “Technical guidelines for safety inspection and evaluation of strains for health food raw materials” (2020 version), and a strain with typical characteristics of Lacticaseibacillus paracasei was selected to obtain a strain of GOLDGUT-Lpc969;

wherein the Gram staining process was as follows:

a single colony of GOLDGUT-Lpc969 was picked out and subjected to bacterial smear, then the bacterial smear was dried, heated and fixed; after crystal violet was dripped for dyeing for 10 s, the bacterial smear was washed with water and spin-dried; after iodine solution was dripped for dyeing for 10 s, the bacterial smear was washed with water and spin-dried; after decolorizing solution was dripped for decolorizing for 10 s, the bacterial smear was washed with water and spin-dried; after sallow solution was dripped for counterstaining for 10 s, the bacterial smear was washed with water; after the bacterial smear was naturally dried, one drop of cedar oil was dripped into the bacteria coating position for oil-microscopic observation, and the observation results were that: GOLDGUT-Lpc969 had a purple Gram stain and was a rod-shaped Gram positive bacterium.

the strain identification process was as follows:

GOLDGUT-Lpc969 was taken and its genome was extracted by a bacterial genome extraction kit, and using a 27F/1492R primer pair (27F: AGAGTTTGATCCGTCTCA, 1492R: TGTACGGY TACCTTGTTACTACGACTT, in 27F and 1492R, M and Y are degenerate bases, M=A or C, Y=C or T) as shown as SEQ ID NO.2 and SEQ ID NO.3, the amplification was performed by using the extracted genome of GOLDGUT-Lpc969 as a template to obtain 16S rRNA of GOLDGUT-Lpc969 (16S rDNA sequence of GOLDGUT-Lpc969 is shown as SEQ ID NO. 1); the 16S rDNA of GOLDGUT-Lpc969 was subjected to nucleic acid sequence alignment in Blastn program of NCBI, and the result showed that the strain was Lacticaseibacillus paracasei, named as Lacticaseibacillus paracasei GOLDGUT-Lpc969, and the Lacticaseibacillus paracasei GOLDGUT-Lpc969 was deposited at the China General Microbiological Culture Collection Center, with a deposit number of CGMCC No. 28149 and a deposit address of No. 3, Courtyard 1, West Beichen Road, Chaoyang District, Beijing;

the physiological and biochemical experimental process was as follows:

a single colony of GOLDGUT-Lpc969 was picked out with a sterile cotton swab, inoculated into sterile saline, and a homogeneous bacterial suspension was prepared by using calibrated VITEK® 2 DensiCHEK™ Plus at a turbidity corresponding to McFarland turbidity of 3.0 and was submitted to physiological and biochemical tests in API20A within 30 min of preparation, which showed that GOLDGUT-Lpc969 could use D-glucose, d-sucrose, D-mannose, d-trehalose, d-maltose, d-arabinose and other substances as carbon sources;

the genome identification analysis procedure was as follows:

GOLDGUT-Lpc969 was taken and its DNA was extracted with SDS method, the quality of extracted DNA was detected by agarose electrophoresis, and DNA quantification was performed by Qubit® 2.0; the genome was sequenced by using Nanopore PromethION platform and Illumina NovaSeq platform, and the sequencing strategy was a 10 Kb library with a sequencing depth of more than or equal to 100×. The sequencing results were: the strain GOLDGUT-Lpc969 contained a circular genome with the size of 2.97 Mbp, and also contained three circular plasmids with the size of 12.88 kbp, 49.33 kbp and 78.09 kbp, respectively. The amino acid sequence encoded by the genome of strain GOLDGUT-Lpc969 was aligned with VFDB database, and the consistency of potential virulence factor sequences obtained through analysis was below 80%, and many of them did not have clear functions. The amino acid sequence encoded by the genome of strain GOLDGUT-Lpc969 was aligned with ARDB database to analyze potential drug resistance genes, and it was found that the consistency with the drug resistance gene sequences was less than 55%. The above results all indicated that the strain GOLDGUT-Lpc969 had high safety;

GOLDGUT-Lpc969 bacterial suspension was prepared as follows:

the Lacticaseibacillus paracasei GOLDGUT-Lpc969 was streaked on MRS solid medium, and cultured at 37° C. for 48 hours to obtain a single colony; the single colony was selected, inoculated in MRS liquid medium, and cultured for 18 hours at 37° C. for activation, so as to obtain an activation solution; the activation solution was inoculated into MRS liquid medium according to the inoculum size of 4% (v/v), and cultured at 37° C. for 18 hours to obtain a bacterial solution; the bacterial solution was centrifuged at 3000 g for 10 min to obtain Lacticaseibacillus paracasei thalli; the Lacticaseibacillus paracasei thalli were washed with 0.9% (m/v, g/100 mL) physiological saline and resuspended in 0.9% (m/v, g/100mL) physiological saline to a cell concentration of 1×10¹⁰ CFU/mL to obtain GOLDGUT-Lpc969 bacterial suspension.

### Embodiment 2

Effect of Lacticaseibacillus paracasei GOLDGUT-Lpc969 on Ovalbumin (OVA)-Induced Hypersensitive Rhinitis Mouse Model

### 1. Experimental Procedure

SPF grade BABL/C female mice aged 6-8 weeks (obtained from Beijing Weitong Lihua Biotechnology Co., ltd., with weight of 18±2 g) were randomly divided into three groups with nine mice in each group, the three groups are: control group (CON), model control group (AR) and probiotic intervention group (Lpc 969) gavaged with GOLDGUT-Lpc969 bacterial suspension.

Mice in the model control group and probiotic intervention group were sensitized on days 0, 7, 14 of the experiment by intraperitoneal injection of 200 μL of OVA suspension (OVA concentration was 1 mg/mL, and the solvent was physiological saline). Mice in probiotic intervention group were gavaged with 200 μL of 1×10¹⁰ CFU/mL GOLDGUT-Lpc969 bacterial suspension (the solvent was 0.9% physiological saline) from day 14 of the experiment, and the control group and the model control group were gavaged with an equivalent of 0.9% physiological saline. On the 21st day of the experiment, the model group and the probiotic intervention group were subjected to nasal drops of 500 μg of OVA daily to create the hypersensitive rhinitis mouse model. Hypersensitive rhinitis symptoms (frequency of nasal scratching, sneezing, and nasal discharge) were recorded within ten minutes after nasal drops in all groups of mice starting at day 28 of the experiment. Day 29 was the endpoint of the experiment and after euthanasia of all mice, inflammation was assessed according to the assessment criteria. The main indexes for evaluating the hypersensitive rhinitis were DAI score, IgE content in mouse serum, histamine content in mouse serum, cytokine content in mouse serum and histopathological section evaluation. DAI scores included nasal scratching times, sneezing times, nasal discharge times, referring to “Zhu Y, Yu J, et al. Experimental observation of the effect of immunotherapy on CD4+ T cells and Th1/Th2 cytokines in mice with allergic rhinitis, Scientific Reports, 2023, 13:5273”.

The DAI score results for all groups of mice at the endpoint of the experiment were shown in FIG. 1.

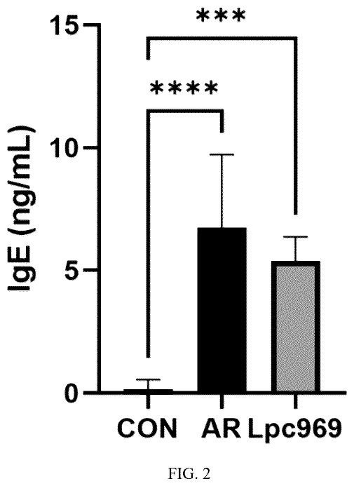

After euthanizing mice at the experimental endpoint, blood of all groups of mice were collected and the serum was isolated, and the content of IgE, histamine and cytokines in serum was detected using a mouse immunoglobulin E (IgE) ELISA kit (No. ml037602, purchased from Shanghai Enzyme-linked Biotechnology Co., Ltd.), a histamine ELISA kit (No. ZK-15029, purchased from Shanghai Zhenke Biotechnology Co., Ltd.), a LEGENDplex MU Th1/Th2 panel (8-plex) cytokine detection kit (No. 741053, purchased from Shenzhen Dakewe Biotech Co., Ltd.), and the statistical results were shown in FIGS. 2 to 10.

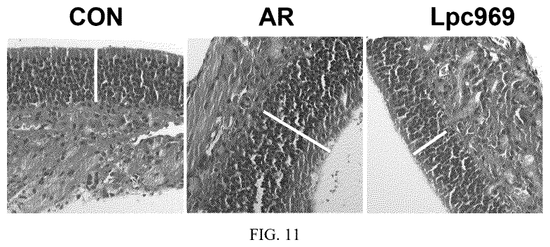

The mice were euthanized at the endpoint of the experiment and the nasal mucosa was removed and fixed with 4% (m/v, g/100 mL) paraformaldehyde; after fixation, paraffin section preparation, H&E staining and PAS staining experiment were carried out, the pathological results of the nasal mucosa were evaluated, and the evaluation results were shown in FIG. 11.

### 2. Experimental Results

The DAI score is one of the important indicators for evaluating rhinitis symptoms of mice, and consists of the frequency of nasal scratching, sneezing and nasal discharge, and the higher the score, the more severe the hypersensitive rhinitis symptoms of mice. As shown in FIG. 1, the DAI score of mice in the model group was significantly increased (to about 2 points) compared to the control group (about 0.2 points), while the DAI score was significantly decreased (to about 1 point) compared to the model group after treatment with the strain GOLDGUT-Lpc969. The results demonstrated that GOLDGUT-Lpc969 could alleviate DAI score increase caused by OVA, and GOLDGUT-Lpc969 can significantly alleviated itching, nasal scratching, sneezing, nasal discharge and other behaviors of mice caused by OVA.

The immune system responds excessively to allergens by producing immunoglobulin E (IgE). Most hypersensitive rhinitis patients show elevated IgE values in blood analysis. Studies have shown that OVA-induced hypersensitive rhinitis causes elevated IgE in mouse serum. As shown in FIG. 2, in this experiment, compared with the control group, the serum IgE in the mice of OVA-induced model group significantly increased (by about 33 times), while the serum IgE in mice treated with GOLDGUT-Lpc969 showed a decreasing trend (by about 20%), demonstrating the potential of GOLDGUT-Lpc969 in treating hypersensitive rhinitis.

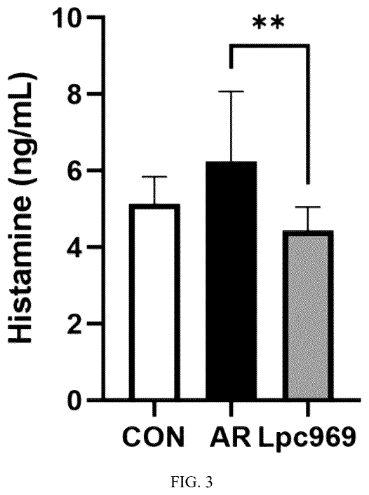

Histamine is involved in local immune and inflammatory reactions. As a center of itching mediators, the histamine content in blood is proportional to the severity of allergic symptoms, and as shown in FIG. 3, the induction of hypersensitive rhinitis by OVA causes the rise of histamine in the serum of mice. After OVA sensitization, the histamine concentration in serum of the mice in model group (AR) increased (by about 21%) compared to the control group (CON). The GOLDGUT-Lpc969 obviously reduced the content of histamine in serum of mice with hypersensitive rhinitis stimulated (by about 30 percent) by OVA, which proved that the strain GOLDGUT-Lpc969 had a certain therapeutic effect on relieving the phenomena of histamine release of the hypersensitive rhinitis and the like.

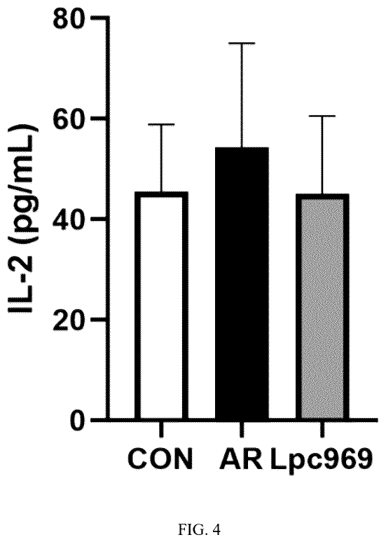

IL-2, IL-4, IL-5, IL-13 in the interleukin (IL) family have been shown to be positively correlated with the severity of hypersensitive rhinitis as a sensitizing factor. As shown in FIG. 4 to FIG. 7, the four cytokines IL-2, IL-4, IL-5 and IL-13 in serum of the mice after OVA stimulation tended to be increased (by about 20%, 200%, 100% and 220%, respectively). After GOLDGUT-Lpc969 treatment, cytokine levels were close to those of the control group and decreased compared to the model group (by approximately 17%, 43%, 59%, and 78%, respectively). It was demonstrated that GOLDGUT-Lpc969 could alleviate symptoms of hypersensitive rhinitis by modulating cytokine levels in serum.

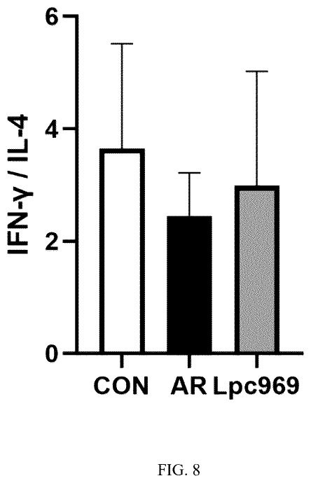

IFN-γ, acting as an important cytokine for Th1 cells, has antagonism with IL-4, so that the activity state of Th1 cells and Th2 cells can be reflected by evaluating the concentration ratio of IFN-γ to IL-4. As shown in FIG. 8, the serum IFN-γ/IL-4 ratio of the mice in the model group showed a decreasing trend (about 33% decrease) compared with that of the control group, and the serum IFN-γ/IL-4 ratio showed a rising trend (about 21% increase) after the treatment with GOLDGUT-Lpc969, and the results demonstrated that the Th1 type immune response activity of the mice was enhanced after the treatment with the GOLDGUT-Lpc969, and the Th2 immune response mediating the allergic reaction was inhibited to some extent, and the allergic symptoms were alleviated.

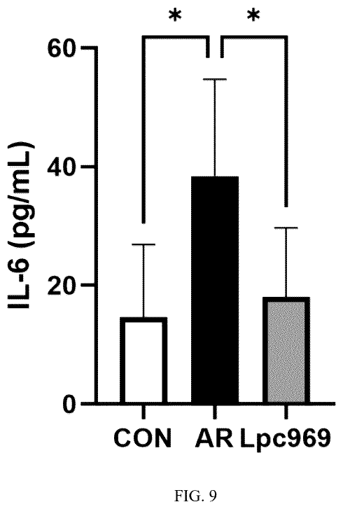

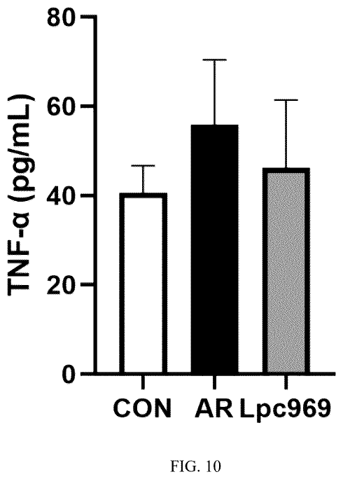

IL-6 and TNF-α have been shown to be positively correlated with inflammation in vivo as a pro-inflammatory factor. As shown in FIG. 9 and FIG. 10, IL-6 was significantly elevated (about 170%) in mouse serum after OVA stimulation, and TNF-α was also prone to increase (about 38%). After the treatment with strain GOLDGUT-Lpc969, the levels of IL-6 and THF-α were reduced (about 53% and 17%, respectively), which proved that the GOLDGUT-Lpc969 could inhibit the inflammation in mice and has positive effect on the treatment of hypersensitive rhinitis.

Compared with normal mice, the pathological section of the nasal mucosa of the mice with hypersensitive rhinitis has the phenomena of cilia shedding, blood vessel congestion, blood vessel hyperplasia, thickening of the nasal mucosa, inflammatory cell infiltration and the like. As shown in FIG. 11, the mice in the control group were normal, the nasal mucosa surface was intact and neat, and no swelling of the mucosa occurred. The mucosal layer of the model group was obviously thickened, and the mucosal surfaces were not orderly arranged. Mice treated with strain GOLDGUT-Lpc969 significantly recovered, the epithelial structure was completely and neatly arranged, and the mucosal thickness returned to a level similar to that of the control group.

### Embodiment 3

Effect of Lacticaseibacillus paracasei GOLDGUT-Lpc969 on Histamine Secretion in RBL-2H3 Cells Induced by Anti-DNP-IgE

### 1. Experimental Procedure

RBL-2H3 cells (purchased from Cell resource center, Institute of Basic Medical Sciences, Chinese Academy of Medical Sciences) were taken out of a liquid nitrogen tank and unfrozen by shaking for 3 min in a water bath at 37° C. to obtain unfrozen RBL-2H3 cells; RBL-2H3 cells were washed with PBS buffer pre-warmed at 37° C. and added with 8 mL of cell medium (the cell medium was a MEM medium containing 10% fetal bovine serum, % referred to volume ratio, the MEM medium was available from Gibco corporation, fetal bovine serum was available from Solarbio corporation), which was cultured at 5% (v/v) CO₂ at 37° C. for 48 h to logarithmic phase; RBL-2H3 cells in logarithmic phase were adjusted to a density value of 5×10⁵ cells/mL using cell medium, and then inoculated in a 24-well plate with 400 μL per well, that is, 2×10⁵ cells per well.

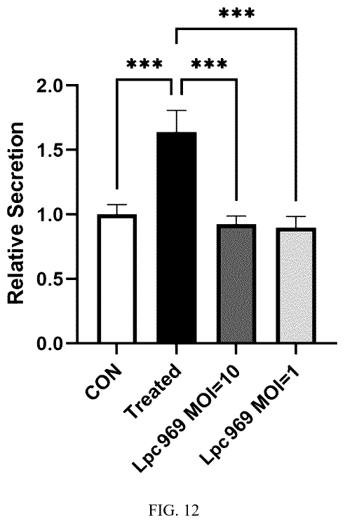

Control wells (CON) were set without any stimulation and 0.2 μg/mL anti-DNP-IgE (purchased from Sigma) was added to the other wells for stimulation at 5% (v/v) CO₂ at 37° C. for 16 hours; after stimulation, PBS buffer was used for washing three times, and it was replaced with fresh MEM medium (purchased from Gibco), treated wells (Treated) without strain interaction were set up and Lpc969 wells with strain GOLDGUT-Lpc969 interaction were set up, with MOI of two groups of 10 and 1 (GOLDGUT-Lpc969 MOI=10, GOLDGUT-Lpc969 MOI=1), and after addition of strain, the interaction was carried out at 5% (v/v) CO₂ at 37° C. for 3 hours; after completion of the interaction, 1 μg/mL DNP-HAS (purchased from 4 ADI Co.) was added to each well for stimulation at 5% (v/v) CO₂ at 37° C. for 15 min; after the stimulation, 100 μL of the cell culture supernatant was pipetted, 20 μL of 1 M NaOH solution and 25 μL of reaction solution were added, and the incubation was carried out for 4 min at room temperature (25° C.) in a dark place; after the incubation, the reaction was terminated by adding 10 μL of 3 M HCl solution; after termination of the reaction, the fluorescence intensity was detected by a multifunctional microplate reader (the excitation wavelength was 355 nm, and the emission wavelength was 460 nm), and histamine levels per well was determined by the formula: relative secretion=OD460ₓ/OD460_{CON,avrg} (in the formula, OD460ₓ was the fluorescence intensity of sample at 460 nm, OD460_{CON,avrg} was the mean value of the fluorescence intensity of CON group at 460 nm), and the experimental results were shown in FIG. 12; wherein, the reaction solution was methanol (purchased from Shenggong Bioengineering (Shanghai) Co., Ltd.) containing 1% (w/v, g/100 mL) o-phthalaldehyde (purchased from Sigma).

### 2. Experimental Results

As shown in FIG. 12, the level of histamine secreted by RBL-2H3 cells in the treated group was significantly higher than that in the control group (about 63% increase), whereas a significant decrease in histamine was observed (about 44% and 46% decrease, respectively) on the premise of MOI of 10 or 1 when treated with Lacticaseibacillus paracasei GOLDGUT-Lpc969. From this, it was found that Lacticaseibacillus paracasei GOLDGUT-Lpc969 reduced the histamine concentration released by anti-DNP-IgE induced RBL-2H3 cells.

### Example 4

Effect of Lacticaseibacillus paracasei GOLDGUT-Lpc969 on Degranulation of RBL-2H3 Cells Induced by anti-DNP-IgE

### 1. Experimental Procedure

RBL-2H3 cells (purchased from Cell resource center, Institute of Basic Medical Sciences, Chinese Academy of Medical Sciences) were taken out of a liquid nitrogen tank and unfrozen by shaking for 3 min in a water bath at 37° C. to obtain unfrozen RBL-2H3 cells; RBL-2H3 cells were washed with PBS buffer pre-warmed at 37° C. and added with 8 mL of cell medium (the cell medium was a MEM medium containing 10% fetal bovine serum, % referred to volume ratio, the MEM medium was available from Gibco corporation, fetal bovine serum was available from Solarbio corporation), which was cultured at 5% (v/v) CO₂ at 37° C. for 48 h to logarithmic phase; RBL-2H3 cells in logarithmic phase were adjusted to a density value of 5×10⁵ cells/mL using cell medium, and then inoculated in a 24-well plate with 400 μL per well, that is, 2×10⁵ cells per well.

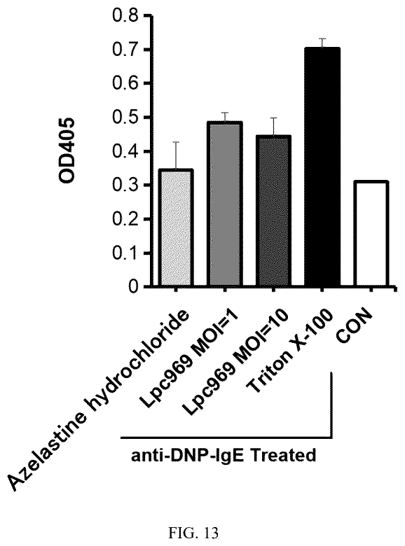

Control wells (CON) were set without any stimulation and 0.2 μg/mL anti-DNP-IgE (purchased from Sigma) was added to the other wells for stimulation at 5% (v/v) CO₂ at 37° C. for 16 hours; after stimulation, PBS buffer was used for washing three times, and it was replaced with fresh MEM medium (purchased from Gibco), positive control wells were set up with 20 μg/mL of the antiallergic agent azelastine hydrochloride (purchased from Michelin Corp.), degranulation complete release groups with 0.1% (v/v) surfactant Triton X-100 (available from Sigma) treatment were set up, and Lpc969 wells with strain GOLDGUT-Lpc969 interaction were set up, the Lpc969 wells were set to two groups with MOI of 10 and 1 (GOLDGUT-Lpc969 MOI=10, GOLDGUT-Lpc969 MOI=1), respectively, and after addition of the strain, the interaction was carried out at 5% (v/v) CO₂ at 37° C. for 3 hours; after completion of the interaction, 1 μg/mL DNP-HAS (purchased from 4ADI Co.) was added to each well for stimulation at 5% (v/v) CO₂ at 37° C. for 15 min; after the stimulation, 25 μL of the cell culture supernatant was pipetted, and an equal volume of matrix solution was added for reaction at 5% (v/v) CO₂ at 37° C. for 1.5 min; after the reaction was completed, 200 μL of a sodium carbonate-sodium hydrogencarbonate buffer solution with a concentration of 0.1 M and pH of 10.0 was added to terminate the reaction; after termination of the reaction, the fluorescence intensity (the excitation wavelength was 355 nm, the emission wavelength was 405 nm) was detected by a multifunctional microplate reader to evaluate the degree of cell degranulation (higher OD₄₀₅ values represented higher degrees of cell degranulation), and the experimental results were shown in FIG. 13; wherein, the matrix solution was a sodium citrate solution with a concentration of 0.2 M and pH of 4.5 containing 5 mM p-nitrophenyl-N-acetyl-ß-d-glucosamide (purchased from national pharmaceutical Co., ltd.) and 5 mM P-nitrophenyl-n-acetylglucosamine (NP-GlcNAc, purchased from national pharmaceutical Co., ltd.).

### 2. Experimental Results

As shown in FIG. 13, the degranulation level of RBL-2H3 cells of Triton X-100 treated group after stimulation was significantly higher than that of the control group (OD₄₀₅ increased by about 0.4), and a significant decrease in the level of cell degranulation was observed when treated with Lacticaseibacillus paracasei GOLDGUT-Lpc969 at MOI of 10 or 1 (OD405 decreased by about 0.25 and 0.2, respectively). From this, it was found that Lacticaseibacillus paracasei GOLDGUT-Lpc969 reduced the degree of degranulation of RBL-2H3 cells induced by anti-DNP-IgE.

Embodiment 5: LPS-induced PBMC cell interaction (cytokine) by Lacticaseibacillus paracasei GOLDGUT-Lpc969

### 1. Experimental Procedure

Human PBMC cells (purchased from Shanghai ORI Biotech Co., Ltd) were taken out of a liquid nitrogen tank and unfrozen by shaking for 2 min in a water bath at 37° C. to obtain unfrozen human PBMC cells; human PBMC cells were washed with PBS buffer pre-warmed at 37° C. and added with 8 mL of cell medium (which was RPMI-1640 medium containing 10% fetal bovine serum inactivated at 56° C. for 30 minutes, % referred to the volume ratio, and RPMI-1640 medium and fetal bovine serum were purchased from Gibco Company), which was cultured at 5% (v/v) CO₂ at 37° C. for 48 h to logarithmic phase; human PBMC cells in logarithmic phase were adjusted to a density value of 1×10⁶ cells/mL using cell medium, and then inoculated in a 96-well plate with 125 μL per well, that is, 8×10⁵ cells per well.

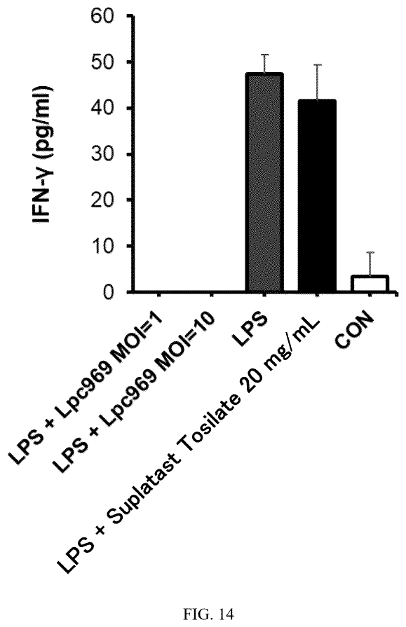

Control wells (CON) were set up without any stimulation, and 5 μg/mL lipopolysaccharide (purchased from Yusheng Biotech Co., ltd.) was added to the other wells for stimulation at 5% (v/v) CO₂ at 37° C. for 2 hours; after stimulation, PBS buffer was used for washing three times, and it was replaced with fresh RPMI-1640 medium (purchased from Gibco), negative control wells (LPS) were set up without strain interaction, drug treatment wells were set up by adding 20 μg/mL of the suplatast tosilate (purchased from Shanghai Aladdin Biochemical Technology Co., Ltd) as control drug, and strain treatment wells were set up by adding strain GOLDGUT-Lpc969 for interaction, which were set to two groups with MOI of 10 and 1 (GOLDGUT-Lpc969 MOI=10, GOLDGUT-Lpc969 MOI=1), respectively, and after addition of the strain, the interaction was carried out at 5% (v/v) CO₂ at 37° C. for 22 hours; after completion of the interaction, centrifugation was performed and the cell culture supernatant was collected, cytokine levels in cell culture supernatants was detected by using a multiplex cytokine kit (LEGENDplex™ HU Th1/Th2 Panel (8-plex) w/VbP V02, from Biolegend, No. 741030), and the remaining cells were added with 10 μL of CCK-8 reagent (from Tongren Chemistry), after the incubation at 5% (V/V) CO₂ at 37° C. for 1 h, centrifugation was performed and the cell culture supernatant was collected, the absorbance was measured at 450 nm, and according to the formula: cell relative viability=(experimental well absorbance value-blank well absorbance value)/(control well absorbance value−blank well absorbance value)×100%, cell relative viability was calculated, and the experiment results were shown in FIG. 14 to FIG. 19.

### 2. Experimental Results

As shown in FIG. 14-FIG. 19, the contents of cytokines such as IFN-γ, IL-10, IL-2, IL-4, IL-6 and TNF-a were significantly increased in the PBMC cell samples of the LPS-treated groups relative to the control groups (IFN-γ, IL-10, IL-2, IL-4, IL-6 and TNF-α were increased by about 43.98, 1.73, 0.09, 486.21 and 703.41, respectively), whereas after the treatment with Lacticaseibacillus paracasei GOLDGUT-Lpc969, a significant decrease in the levels of the above cytokines could be observed at MOI of 10 or 1 (at MOI of 10, IFN-γ, IL-10, IL-2, IL-4, IL-6 and TNF-α decreased by approximately 47.37, 1.77, 0.94, 3.52, 557.81 and 773.58 respectively compared with the LPS-treated groups, and at MOI of 1,IFN-γ, IL-10, IL-2, IL-4, IL-6 and TNF-α decreased by approximately 47.37, 1.77, 0.94, 3.60, 557.81 and 773.58 respectively compared with the LPS-treated group), and the performance was better than that of the positive control drug sutemilast tosilen (IFN-γ, IL-10, IL-2, IL-4, IL-6 and TNF-α decreased by approximately 5.80, −0.12, 0.04, 0.75, −84.55 and 70.07 respectively compared with the LPS-treated groups). From this, it was found that the Lacticaseibacillus paracasei GOLDGUT-Lpc969 significantly inhibited the release of inflammatory cytokines induced by LPS.

It is apparent that the descriptions of the above embodiments are only examples for clear explanation and are not limitations on embodiments. Other variations or modifications of the above teachings will be apparent to those of ordinary skill in the art. It is not necessary here nor is it exhaustive of all embodiments. And obvious variations or modifications introduced herein are still within the scope of the present disclosure.

What is claimed is:

1. A strain of Lacticaseibacillus paracasei, wherein the strain of Lacticaseibacillus paracasei was deposited in China General Microbiological Culture Collection Center (CGMCC) with a deposit number of CGMCC No. 28149.

2. An application of the strain of Lacticaseibacillus paracasei according to claim 1 for preparing a drug for a prevention and/or a treatment of hypersensitive rhinitis.

3. The application according to claim 2, wherein the prevention and/or the treatment of hypersensitive rhinitis comprises alleviation of symptoms of hypersensitive rhinitis, inhibition of immune responses caused by hypersensitive rhinitis, inhibition of inflammatory responses caused by hypersensitive rhinitis and/or repair of lesions of nasal mucosa caused by hypersensitive rhinitis.

4. The application according to claim 3, wherein the alleviation of symptoms of hypersensitive rhinitis comprises reducing the frequency of nasal scratching, sneezing and/or nasal discharge in patients with hypersensitive rhinitis.

5. A product comprising the strain of Lacticaseibacillus paracasei of claim 4.

6. The product according to claim 5, wherein the product comprises a food or a drug.

7. The product according to claim 6, wherein the drug comprises the strain of Lacticaseibacillus paracasei, a drug carrier and/or a pharmaceutical adjuvant.

8. The product according to claim 7, wherein the drug carrier comprises a microcapsule, a microsphere, a nanoparticle and/or a liposome.

9. The product according to claim 7, wherein the pharmaceutical adjuvant comprises an excipient and/or an additive.

10. The product according to claim 9, wherein the additive comprises microcrystalline cellulose, hydroxypropyl methylcellulose and/or refined lecithin.


SEQUENCE LISTING (3 sequences)

SEQ ID NO: 1 - DNA, 1463 bp, synthetic construct
  source 1..1463: mol_type = other DNA; note = Lacticaseibacillus paracasei
tgatgggtgcctatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagcagtctcacgcg

SEQ ID NO: 2 - DNA, 19 bp, synthetic construct
  source 1..19: mol_type = other DNA; note = primer
agagtttgatcmtggctca

SEQ ID NO: 3 - DNA, 24 bp, synthetic construct
  source 1..24: mol_type = other DNA; note = primer
tgtacggytaccttgttacgactt